(19) United States
(12) Patent Application Publication
(10) Pub. No.: US 2006/0123496 A1
(43) Pub. Date: Jun. 8, 2006

(54) Animal models

(75) Inventors: Edward Duncan Blair, Hertfordshire (GB); Neil James Clarke, Hertfordshire (GB); Sebastian Lennox Johnston, London (GB); David John Rowlands, Yorkshire (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Glaxo Group Limited

(21) Appl. No.: 10/521,319

(22) PCT Filed: Jul. 17, 2003
(86) PCT No.: PCT/EP03/07939

§ 371 Date: Dec. 1, 2005

(30) Foreign Application Priority Data
Jul. 18, 2002 (GB) 0216729.4

(51) Int. Cl. A01K 67/027

(52) U.S. Cl. 800/14; 800/18

(57) ABSTRACT

A transgenic non-human animal whose genome comprises a polynucleotide encoding human ICAM-1 domains D1 and D2 and preferably encoding human ICAM-1 domains D1 and D2 and host non-human ICAM-1 domains D3, D4 and D5. Methods for the production of such transgenic animals, screening methods using such transgenic animals, associated transgenes, constructs, vectors, and cells are disclosed. The transgenic non-human animal is a suitable model for studying human rhinovirus infection.

### FIELD OF THE INVENTION

The present invention relates to a non-human animal model suitable for studying human rhinovirus (HRV) infection. In particular the invention relates to a non-human animal model for screening potential agents for the treatment of HRV infection, such as the common cold. The invention also relates to associated polynucleotide transgenes, constructs, vectors, cells and to methods for testing and screening potential drugs.

### BACKGROUND OF THE INVENTION

Human rhinovirus (HRV) infections cause two thirds of upper respiratory tract infections (common colds) which are associated with enormous morbidity, absence from school and work and health care costs. Lower respiratory tract HRV infections are also associated with life threatening illnesses such as pneumonias in the immunosuppressed, acute exacerbations of asthma, (Johnston, S. L., Allergy 53,922-932 (1998) and other respiratory diseases such as chronic obstructive pulmonary disease (COPD; Seemungal, T., et.al., Am.J.Respir.Crit Care Med. 164,1618-1623, 2001). Asthma now affects up to 30% of children and 10-15% of adults in the westernised communities and HRV infection is implicated in the majority of acute exacerbations of the disease, (Johnston, S. L., et.al., BMJ, 310, 1225-1229, 1995). The overall morbidity and economic burden attributable to HRV is therefore considerable.

HRVs constitute the majority of the rhinovirus genus of the Picomaviridae. There are over 100 distinct serotypes of HRV and this has prevented the development of effective vaccines. A small animal model of HRV infection would be invaluable for testing potential anti rhinoviral compounds and for elucidating mechanisms of disease to generate new targets for development of therapies. However, there is no small animal model for HRV infection and in vivo research is restricted to large primate studies or studies in human volunteers.

A major obstacle to the development of small animal models of infection, such as murine models, is the host cell tropism of HRV. Approximately 10% of HRV serotypes make up the minor receptor group, which can use both the human and animal, such as murine, forms of low-density lipoprotein receptor (LDLr) to enter cells of either species. However, the remaining 90% of HRV's comprise the major receptor group and use human intercellular adhesion molecule-1 ( ICAM-1) to achieve cell attachment and entry (Greve, J. M., et. al, Cell, 56, 839-847, 1989; Staunton, et. al, Cell, 56,849-853,1989; Tommassini, J. E., et.al/, Proc.Natl. Acad. Sci. USA 86, 4907-4911,1989; Uncapher,C. R., et. al., Virology, 180, 814-817,1991). These viruses do not appear to bind to non-human particularly murine ICAM-1 (e.g. Register, R. B., et.al., J.Virol., 65, 6589-6596,1991; Staunton, D. E., et.al., J.mmunol. 148, 3271-3274, 1992) and therefore species specific restriction of HRV replication is implemented at the earliest stage in the infection cycle.

ICAM-1 (CD54) is involved in inflammatory functions mediated by leukocyte adhesion(Makgoba, M. W., et.al., Nature, 331, 86-88, 1988). It belongs to the immunogobulin supergene family and both human and non-human e.g. murine ICAM-1 have five homologous ‘Ig-like’ extracellular domains (D1 to D5), a transmembrane domain and a short C-terminal cytoplasmic domain. The two amino-terminal domains of human ICAM-1 (D1 and D2) have been shown to bind to major group HRV's and to confer this ability on a murine ICAM-1 polypeptide (Register, R. B., et al, 1991; J. Virol. 65, 6589-6596; Staunton, D. E., et.al., Cell, 61, 243-254, 1990 Bella et aL, Proc.Natl.Acad.Sci.USA,95:4140, 1998; Staunton, D. E., et.al., J.lmmunoL 148, 3271-3274,1992). The transmembrane and cytoplasmic domains of ICAM-1 are not thought to be required for internalization of major group HRVs but may be required for normal ICAM-1 intracellular signalling functions (Staunton, D. E., et.al., J.Immunol. 148,3271-3274,1992).

Despite increased knowledge of HRV and mechanisms of infection, there remains a critical need to develop a small animal model of HRV infection.

### SUMMARY OF THE INVENTION

The present inventors have generated a transgenic animal model of HRV respiratory tract infection. They have found that the use of a construct comprising a polynucleotide sequence which encodes human ICAM-1 domains D1 and D2 results in a transgenic non-human animal that expresses a ICAM-1 polypeptide which is capable of supporting binding and infection of major group HRV. Furthermore, the transgenic non-human animals of the invention are particularly capable of expressing the ICAM-1 polypeptide in cells and tissues of the respiratory tract.

Accordingly, the present invention provides a transgenic non-human animal whose genome comprises a polynucleotide encoding human ICAM-1 domains D 1 and D2. The transgenic non-human animal of the invention is capable of expressing a ICAM-1 polypeptide which comprises human ICAM-1 domains D 1 and D2.

In one embodiment, the present invention provides a transgenic non-human animal whose genome comprises a polynucleotide encoding human ICAM-1 domains D 1 and D2 and one or more of host non-human animal ICAM-1 domains D3, D4 and D5 and most preferably a polynucleotide encoding human ICAM-1 domains D 1 and D2 and each of host non-human animal ICAM-1 domains D3, D4 and D5. Additionally the polynucleotide incorporated into the animal genome may encode host non-human animal ICAM-1 transmembrane and/or cytoplasmic domains. The transgenic animal is capable of expressing a chimaeric ICAM-1 polypeptide which comprises human ICAM-1 domains D 1 and D2 and one or more of host non-human animal ICAM-1 domains D3, D4 and D5, and preferably a chimaeric ICAM-1 polypeptide which comprises human ICAM-1 domains D 1 and D2 and each of host non-human animal ICAM-1 domains D3, D4 and D5. The chimaeric ICAM-1 polypeptide may further comprise host non-human animal transmembrane and/or cytoplasmic domains of ICAM-1.

The transgenic non-human animal of the invention provides a small animal model of HRV respiratory tract infection. Such a model provides an extremely useful tool for identifying and evaluating (with regard to efficacy and safety) therapeutic agents e.g. chemical compounds which inhibit or otherwise modulate HRV binding, internalisation and/or replication, and furthermore would assist in further defining mechanisms of viral immunopathology and evaluating potential therapeutic targets. Additionally, such a model could usefully be combined with existing small animal models of asthma and COPD to study the role of viral infection in the exacerbation of airway disease.

### BRIEF DESCRIPTION OF THE SEQUENCE LISTINGS AND FIGURES

SEQ ID No:1 provides the polynucleotide (DNA) sequence encoding human ICAM-1 polypeptide

SEQ ID No:2 provides the polynucleotide (DNA) sequence encoding human ICAM-1 domain D1

SEQ ID No:3 provides the polynucleotide (DNA) sequence encoding human ICAM-1 domain D2

SEQ ID No:4 provides the polypeptide (amino acid) sequence of human ICAM-1

SEQ ID No:5 provides the polypeptide (amino acid) sequence of human ICAM-1 domain D1

SEQ ID No:6 provides the polypeptide (amino acid) sequence of human ICAM-1 domain D2

SEQ ID No:7 provides the polynucleotide (DNA) sequence encoding murine ICAM-1 polypeptide

SEQ ID No:8 provides the polynucleotide (DNA) sequence encoding murine ICAM-1 domain D3

SEQ ID No:9 provides the polynucleotide (DNA) sequence encoding murine ICAM-1 domain D4

SEQ ID No:10 provides the polynucleotide (DNA) sequence encoding murine ICAM-1 domain D5

SEQ ID No:11 provides the polypeptide (amino acid) sequence of murine ICAM-1 domain D3

SEQ ID No:12 provides the polypeptide (amino acid) sequence of murine ICAM-1 domain D4

SEQ ID No:13 provides the polypeptide (amino acid) sequence of murine ICAM-1 domain D5

SEQ ID No:14 provides the nucleotide sequence (DNA) encoding a human/mouse chimaeric ICAM-1 polypeptide

SEQ ID No:15 provides the polypeptide (amino acid) sequence of a human /mouse chimaeric ICAM-1 polypeptide

SEQ ID No:16 provides the nucleotide sequence (DNA) encoding rat ICAM-1 polypeptide

FIG. 1: This shows the plasmid vector pcDNA3.1+ containing a human-mouse chimaeric ICAM-1 cDNA

FIG. 2: This demonstrates that murine respiratory epithelial cells (LA4) transfected with a human-mouse chimaeric ICAM-1 DNA express chimaeric ICAM-1

FIG. 3: This demonstrates that murine respiratory epithelial cells (LA4) expressing chimaeric ICAM-1 are susceptible to infection with and can support the replication of major group HRV

FIG. 4: This demonstrates the replication of major group HRV in murine respiratory epithelial cells

FIG. 5: This shows details of human ICAM-1 peptides used for generation of rabbit anti-human ICAM-1 polyclonal antibodies

FIG. 6: This demonstrates the expression of chimaeric ICAM-1 in airways tissue of transgenic mice

FIG. 7: This shows HRV-induced inflammatory cytokine production and cell necrosis in primary tracheal cells isolated from transgenic mice

### DETAILED DESCRIPTION OF THE INVENTION

The present invention provides a transgenic non-human animal whose genome comprises a polynucleotide encoding a ICAM-1 polypeptide comprising human ICAM-1 domains D1 and D2, preferably human ICAM-1 domains D 1 and D2 and one or more of host non-human animal ICAM-1 domains D3, D4 and D5, and most preferably all three host non-human animal ICAM-1 domains D3,D4 and D5. The ICAM-1 polypeptide encoded by the polynucleotide incorporated into the non-human animal genome may further comprise host non-human animal ICAM-1 transmembrane and/or cytoplasmic domains. The transgenic non-human animal is capable of expressing a chimaeric ICAM-1 polypeptide. The expressed ICAM-1 polypeptide such as a chimaeric ICAM-1 polypeptide is at least capable of supporting binding and infection of major group HRV and is expressed particularly in cells and/or tissues of the respiratory tract or airways. Preferably, the ICAM-1 polypeptide has the basic biological functionality of native ICAM-1 of the host animal. Such basic biological functionality would be appreciated by a person skilled in the art to include without limitation any of the following cell-cell adhesion in inflammatory and immune systems, where ICAM-1 mediates responses such as leukocyte attachment to endothelium and migration towards sites of inflammation. It may also include, T and B cell activation, thymocyte development, and T-cell effector function. In addition, ICAM-1 functions in cell-cell signalling, for example in T-cell activation as a costimulatory molecule and as an inducer of apoptosis.

The polynucleotide comprised in the genome of the transgenic non-human animal may comprise any number of nucleic acid sequences. For example, the polynucleotide may comprise one or more nucleic acid sequences encoding human domains D1 and D2 and/or one or more nucleic acid sequences encoding non-human domains D3, D4 and D5. In all aspects, however, the polynucleotide preferably encodes a ICAM-1 polypeptide e.g. chimaeric ICAM-1 which is capable of being expressed in the non-human animal, particularly in airways tissues.

The polynucleotide comprised in the genome of the transgenic non-human animal includes wild type forms and any modified forms provided the expressed chimaeric ICAM-1 is capable of binding and supporting infection with major group HRV. Modified polynucleotides include without limitation those which have one or more nucleotide substitutions, deletions, insertions or inversions.

In a preferred aspect the transgenic non-human animal is a mammal, preferably a rodent such as a mouse, rat or guinea pig, and more preferably a mouse. Thus, in a particularly preferred embodiment the chimaeric ICAM-1 polypeptide comprises human ICAM-1 domains D1 and D2 and one or more of murine ICAM-1 domains D3, D4 and D5, especially human ICAM-1 domains D1 and D2 and each of murine ICAM-1 domains D3, D4 and D5.

The polynucleotide encoding the ICAM-1 polypeptide comprised in the genome of a transgenic non-human animal of the invention may comprise one or more of the following polynucleotide sequences:

(a) a polynucleotide sequence having at least 90%, more preferably 95%, 96%, 97%, 98%, 99% or 100% identity to the polynucleotide sequence of SEQ ID No:2;

(b) a polynucleotide sequence having at least 90%, more preferably 95%, 96%, 97%, 98%, 99% or 100% identity to the polynucleotide sequence of SEQ ID No:3;

(c) a polynucleotide sequence encoding a polypeptide sequence having at least 90%, more preferably 95%, 96%, 97%, 98%, 99% or 100% identity to the polypeptide sequence of SEQ ID No:5;

(d) a polynucleotide sequence encoding a polypeptide sequence having at least 90%, more preferably 95%, 96%, 97%, 98%, 99% or 100% identity to the polypeptide sequence of SEQ ID No:6;

(e) a polynucleotide fragment of SEQ ID No:1 (or a sequence with at least 90%, or more preferably 95%, 96%, 97%, 98%, 99% or 100% identity to it) encoding human ICAM-1 domains D1 and D2; and

(f) a polynucleotide sequence encoding a polypeptide fragment of SEQ ID No:4 (or a sequence with at least 90%, more preferably 95%, 96%, 97%, 98%, 99% or 100% identity to it) comprising human ICAM-1 domains D1 and D2.

When the transgenic non-human animal is a mouse, the polynucleotide encoding the chimaeric ICAM-1 comprised in the mouse genome may comprise in addition to the human nucleotide sequences defined above one or more of the following murine polynucleotide sequences:

(a) a polynucleotide sequence having at least 90%, more preferably 95%, 96%, 97%, 98%, 99% or 100% identity to the polynucleotide sequence of SEQ ID No:8;

(b) a polynucleotide sequence having at least 90%, more preferably 95%, 96%, 97%, 98%, 99% or 100% identity to the polynucleotide sequence of SEQ ID No:9;

(c) a polynucleotide sequence having at lease 90%, more preferably 95%, 96%, 97%, 98%, 99%, or 100% identity to the polynucleotide sequence of SEQ ID No:10;

(d) a polynucleotide sequence encoding a polypeptide sequence having at least 90%, more preferably 95%, 96%, 97%, 98%, 99% or 100% identity to the polypeptide sequence of SEQ ID No:11;

(e) a polynucleotide sequence encoding a polypeptide sequence having at least 90% or more preferably 95%, 96%, 97%, 98%, 99% or 100% identity to the polypeptide sequence of SEQ ID No:12;

(f) A polypeptide sequence encoding a polypeptide sequence having at least 90%, more preferably 95%, 96%, 97%, 98%, 99%, or 100% identity to the polypeptide sequence of SEQ ID No:13; and

(g) a polynucleotide fragment of SEQ ID No:7 (or a sequence with at least 90%, more preferably 95%, 96%, 97%, 98%, 99% or 100% identity to it) encoding one or more of murine ICAM-1 domains D3, D4 and D5.

In one particular embodiment of a transgenic mouse of the invention, the polynucleotide encoding a chimaeric ICAM-1 polypeptide comprised in the mouse genome comprises one or more of the following polynucleotide sequences:

(a) a polynucleotide sequence having at least 90%, more preferably 95%, 96%, 97%, 98%, 99% or 100% identity to the polynucleotide sequence of SEQ ID No:14.

(b) a polynucleotide encoding a polypeptide having at least 90%, more preferably 95%, 96%, 97%, 98%, 99% or 100% identity to the polypeptide sequence of SEQ ID No:15.

When the transgenic non-human animal is a rat, the polynucleotide encoding the chimaeric ICAM-1 comprised in the rat genome may comprise a polynucleotide sequence comprising a fragment of SEQ ID No:16 (or a sequence at least 90%, more preferably 95%, 96%, 97%, 98%, 99% or 100% identity to it) encoding one or more of rat ICAM-1 domains D3, D4 and D5.

The ICAM-1 polypeptide expressed by the non-human transgenic animal may comprise one or more of the following polypeptide sequences:

(a) a polypeptide fragment of SEQ ID No:4 (or a sequence with at least 90%, more preferably 95%, 96%, 97%, 98%, 99% or 100% identity to it) comprising human ICAM-1 domains D1 and D2;

(b) a polypeptide sequence having at least 90%, more preferably 95%, 96%, 97%, 98%, 99% or 100% identity with the polypeptide sequence of SEQ ID No:5; and

(c) a polypeptide sequence having at least 90%, more preferably 95%, 96%, 97%, 98%, 99% or 100% identity with the polypeptide sequence of SEQ ID No:6.

The chimaeric ICAM-1 polypeptide encoded by the polynucleotide incorporated into the genome of a non-human animal may further comprise a polypeptide comprising one or more of host non-human ICAM-1 domains D3, D4 and D5, and preferably all three.

When the transgenic non-human animal is a mouse the human/murine chimaeric ICAM-1 polypeptide encoded by the polynucleotide incorporated into the mouse genome may comprise one or more of the following polypeptide sequences:

(a) a polypeptide fragment of SEQ ID No:7 (or a sequence with at least 90%, more preferably 95%, 96%, 97%, 98%, 99% or 100% identity to it) comprising one or more of murine ICAM-1 domains D3, D4 and D5;

(b) a polypeptide sequence having at least 90%, more preferably 95%, 96%, 97%, 98%, 99% or 100% identity with the polypeptide sequence of SEQ ID No: 11;

(c) a polypeptide sequence having at least 90%, more preferably 95%, 96%, 97%, 98%, 99% or 100% identity with the polypeptide sequence of SEQ ID No:12; and

(d) a polypeptide sequence having at least 90%, more preferably 95%, 96%, 97%, 98%, 99% or 100% identity with the polypeptide sequence of SEQ ID No:13.

In a preferred embodiment, the chimaeric ICAM-1 polypeptide comprises human ICAM-1 domains D1 and D2 and murine ICAM-1 domains D3, D4 and D5. Thus in a particular embodiment, the human/murine chimaeric ICAM-1 polypeptide comprises:

(a) a polypeptide sequence having at least 90%, more preferably 95%, 96%, 97%, 98%, 99% or 100% identity to the polypeptide sequence of SEQ ID No:15.

The chimaeric ICAM-1 polypeptide is expressed in the cells and tissues of the non-human transgenic animal and is particularly expressed in the cells and/or tissues of the respiratory tract or airways, especially respiratory tract epithelial cells.

The present invention relies on the use of a nucleic acid construct or transgene to generate transgenic animals of the present invention. The construct or transgene comprises a polynucleotide encoding human ICAM-1 domains D1 and D2, one or more of host non-human (such as rat or mouse) ICAM-1 domains D3, D4 and D5 as defined herein (and optionally host non-human ICAM-1 transmembrane and/or cytoplasmic domains), and a suitable regulatory sequence such as a promoter which can be expressed in a broad range of different cell types including cells and/or tissues of the respiratory tract, for example, a CMV or SV40 promoter. Also suitable are promoters with an airways-restricted pattern of expresson, such as the human surfactant protein C (SPC) or Clara cell 10-kDa secretory protein (CC10) promoter.

The assembly of the transgenic construct follows standard cloning techniques, that are well known in the art (for example see Sambrook, et al., Molecular Cloning: A labatory Manual, 2nd Ed., Cold Spring Harbor Laboratory Press, Cold Spring Harbor., N.Y. (1989).

The polynucleotide encoding chimaeric ICAM-1 may be constructed from cDNA or genomic DNA of ICAM-1 from a human and non-human animal. If it is cDNA, the cDNA can be prepared de novo by e.g reverse transcriptase PCR (RT-PCR) from an mRNA extracted from a relevant tissue of a human and of a non-human animal. Alternatively, the cDNA may be excised from vectors containing the full length cDNAs of human or murine ICAM-1. The chimaeric cDNA, together with the desired regulatory sequence and any other desired components such as artificial introns, polyadenylation sequences, reporter genes and genes which will allow antibiotic selection in both prokaryotic (e.g bacterial) and mammalian cell lines can then be inserted into a cloning vector by restriction digest and ligation. Suitable vectors such as plasmids for the assembly of transgenes are those which provide for acceptable yields of DNA. Typical examples could include, pCDNA 3.1, pBlueScript, or other plasmid cloning vectors having a medium to high copy number in routinely used strains of Escherichia coli.

The regulatory sequences, such as promoters, are operably linked to the coding sequence of the chimaeric ICAM-1 encoding polynucleotide or polynucleotides in a manner that will permit expression of the polynucleotide. There may or may not be intervening sequences between the chimaeric ICAM-1 encoding polynucleotide(s) and the promoter, provided that the promoter directs expression of the desired polynucleotides appropriately.

Methods of linking regulatory sequences to cDNAs to facilitate their expression are widely known in the art. Such methods include without limitation directly ligating a nucleic acid sequence comprising a regulatory sequence to the coding region of the desired polynucleotide in this case the polynucleotide encoding chimaeric ICAM-1. Additional nucleic acid sequences may be included that modulate expression and include enhancer elements, artificial introns and others.

Preferably prior to the introduction of the chimaeric transgene into the host cell, the vector portions are removed by restriction enzyme digestion, for example by using restriction sites in the vector that flank the transgene. Thus the genetic material introduced into a host cell will preferably comprise the polynucleotide encoding chimaeric ICAM-1 as defined herein and the regulatory sequences to which it has been operably linked together with other potential components of the transgene such as polyadenylation sequences, reporter genes and genes which will allow antibiotic selection in the cell.

The present invention also includes any cells cultured from the transgenic non-human animals of the invention. The cells are cultured in-vitro. The genome of the cells thus comprise the construct or transgene of the invention. Preferred cells are cells of the respiratory tract implicated or otherwise involved in HRV infection such as respiratory epithelial cells.

Cells cultured in-vitro from a transgenic animal may be prepared by any suitable method. The cells are typically rodent such as mouse or rat, and most preferably mouse. Cultures of respiratory cells such as respiratory epithelial cells can therefore be provided and used for in-vitro experiments such as in-vitro screening of test agents that may modulate HRV binding and infection.

The invention also includes any cell transformed or transfected preferably stably transfected with a polynucleotide encoding chimaeric ICAM-1 as defined herein. Such cells include bacterial and yeast cells used to replicate a vector comprising the chimaeric ICAM-1 encoding polynucleotide, non-human cells such as respiratory epithelial cells to generate a cell line useful for relevant in-vitro studies and stem cells that are used to generate the non-human transgenic animals of the invention. The non-human transgenic animals of the invention may be generated by the use of any suitable protocol known in the art such as:

introducing the desired polynucleotide into a suitable cell

allowing the cell to develop into a transgenic animal

optionally, breeding the animal true.

There are a number of techniques that permit the introduction of genetic material, such as a transgene, into the germline. A commonly used, protocol comprises direct injection of the transgene into the male pronucleus of the fertilised egg (Hogan et al., Manipulating the mouse embryo (A laboratory manual) Second edition, CSHL Press 1994), resulting in the random integration into one locus of a varying number of copies, usually in a head to tail array (Costantini and Lacy, Nature 294, 92, 1981). The injected eggs are then re-transferred into the uteri of pseudo-pregnant recipient mothers. Some of the resulting offspring may have one or several copies of the transgene integrated into their genomes, usually in one integration site. These “founder” animals are then bred to establish transgenic lines and to back-cross into the genetic background of choice. It is convenient to have the transgene insertion on both chromosomes (homozygosity) as this obviates the need for repeated genotyping in the course of routine mouse husbandry.

Alternatively, for the production of transgenic mice, transgenes can be introduced via embryonic stem (ES) cells, using electroporation, retroviral vectors or lipofection for gene transfer. This is followed by the random insertion into the genome of the pluripotent embryonic stem (ES) cells, followed by the production of chimaeric mice and subsequent germline transmission. Transgenes of up to several hundred kilobases of rodentian DNA have been used to produce transgenic mice in this manner (for example Choi et al., Nature Genet. 4,117-123 (1993); Strauss et al., Science 259, 1904-07 (1993)). The latter approach can be tailored such that the transgene is inserted into a pre-determined locus (non-randomly, for example ROSA26 or HPRT) that supports ubiquitous as well as tissue specific expression of the transgene (Vivian et al., BioTechniques 27, 154-162 (1999)). The transgenic animals can be subsequently tested to ensure the required genotypic change has been effected, in any suitable fashion. This can be done by, for example, detecting the presence of the transgene by PCR with specific primers, or by Southern blotting of tail DNA with a specific probe. Testing for homozygosity of the transgene insertion may be carried out using quantitative Southern blotting to detect a twofold difference in signal strength between hetero- and homozygous transgenic animals. Once the desired genotype has been confirmed the transgenic animal line can be subjected to various tests to determine the phenotype. The tests involved in this phenotypic characterisation depend on what genotypic change has been effected, and may include, for example, morphological, biochemical and behavioural studies. The transgenic animals of the present invention demonstrate expression of chimaeric ICAM-1 as defined herein and hence the ability to support binding, internalisation and infection of major group HRV.

Expression of a chimaeric ICAM-1 polypeptide can be established at the RNA or protein level e.g. by obtaining the corresponding mRNA or protein from the animal and analysing expression. Infection by major group HRV can be determined by for example observing cytopathic effects in relevant cells such as respiratory epithelial cells. Infection may also be determined by virus plaque assay using lavage samples obtained from the upper or lower respiratory airways of the infected animal. The use of non-human animal models with altered expression of more than one gene can provide important insights into the genetic basis of particular diseases for example asthma and other inflammatory respiratory disorders. Consequently the interbreeding of the transgenic non-human animals of the present invention with non-human animal models which overexpress, underexpress or have an otherwise altered expression of a different gene may produce superior animal models of diseases such as asthma. In particular, such an approach could be used to develop a superior animal model in which the role of viral infection e.g. HRV infection in the exacerbation of respiratory disorders such as asthma and COPD could be studied.

Additionally, the transgenic non-human animal model of the present invention can be used to study the role of HRV infection in excaberation of asthma or other airways disease in suitable animal models. In the case of asthma, the animal is “primed” and sensitised by repeated injection of ovalbumin over time, followed by subsequent exposure (“challenge”) of particulate albumin in the airways, as described ( Lloyd, C. M., et.al., Advances in Immunology. 77, 263-95, 2001). Measurements of lung function would indicate the severity of the disease in the presence or absence of other exacerbating factors such as HRV.

Cells and animals of the present invention may also be used to identify and test the efficacy of potential therapeutic agents for the treatment of conditions associated with HRV infection. Potential therapeutic agents may inhibit (antagonise) major group HRV binding to the chimaeric ICAM-1 expressed by the transgenic animals of the invention, or inhibit (antagonise) viral replication.

A method of identifying a therapeutic agent for the treatment of conditions associated with or exacerbated by major group HRV infection can therefore be provided, comprising:

infecting a transgenic animal of the invention with major group HRV,

administering to a transgenic animal of the invention a test substance, and

determining whether the test substance (i) prevents or delays the onset of the condition or (ii) treats or alleviates the condition.

Conditions associated with major group HRV infection include the common cold and conditions exacerbated by major group HRV infection include asthma, COPD and other respiratory disorders. Thus the efficacy of a test substance may be determined by analysing cellular e.g. cytopathic effects, plaque assay and/or physiological e.g. exacerbation of typical adverse respiratory symptoms changes following HRV Infection.

The invention further provides a method of identifying a therapeutic agent for the treatment of a condition associated with or exacerbated by major group HRV infection, comprising:

infecting a cell of the invention with major group HRV,

contacting a test substance with a cell of the invention, and

determining whether the test substance (i) prevents or delays the onset of cellular changes associated with the condition or (ii) causes a decrease in any of the cellular changes associated with the condition.

In the aforementioned screening methods, the transgenic animal or cell line as appropriate may be infected with major group HRV prior to or subsequent to administering or contacting with a test substance. Typically however the transgenic animal or cell line will be infected with major group HRV prior to administering or contacting with a test substance. In the screening method employing a transgenic animal of the invention the virus may be administered to the animal via the tracheal or nasal routes at a virus concentration sufficient to cause infection, such as concentrations of about 10⁶ to 10⁹ viral particles/mouse, for example, about 10⁸ viral particles/mouse.

To obtain suitable virus infection levels, it may be desirable to use a temperature adapted major group HRV, that is a virus strain adapted to grow optimally within mouse upper and lower respiratory airways. Such a virus strain can be prepared by routine methods which may involve, for example, serially passaging a strain of major group HRV alternately between human and mouse epithelial cell cultures for up to 50 passages, optionally at increasing temperatures up to the normal temperature of mouse upper and lower respiratory airways i.e. passaging from about 33° C. to about 40° C., for example from about 35° C. to about 40° C.

Suitable test substances which may be tested in the above methods include antibody products (for example, monoclonal and polyclonal antibodies, single chain antibodies, chimaeric antibodies and CDR grafted antibodies). Furthermore, combinatorial libraries, defined chemical identities, small molecules, peptide and peptide mimetics, oligonucleotides and natural product libraries, such as display libraries (e.g. phage display libraries) may also be tested. Preferably, the test substances may be chemical compounds. Known anti-viral agents that inhibit HRV infection and replication include, for example, pleconaril (capsid function inhibitor), AG7088 (viral 3C protease inhibitor) and recombinant soluble intercellular adhesion molecule (sICAM-1). Batches of the test substances may be used in an initial screen of, for example, ten substances per reaction, and the substances of batches which show inhibition tested individually.

Agents identified in the screening methods of the invention may be used to prevent or treat the conditions discussed above. The clinical outcome of a patient suffering from such a disease can therefore be improved by administration of such a product.

The formulation of the product for use in preventing or treating any condition will depend upon factors such as the nature of the agent identified and the disease to be prevented or treated. Typically the agent is formulated for use with a pharmaceutically acceptable carrier or diluent. For example it may be formulated for inhaled, intracranial, parenteral, intravenous, intramuscular, subcutaneous, transdermal or oral administration but particularly for inhaled and oral administration. A physician will be able to determine the required route of administration for each particular patient.

The dose of product may be determined according to various parameters, especially according to the substance used; the age, weight and condition of the patient to be treated; the route of administration; and the required regimen. A suitable dose may however be from 0.1 to 100 mg/kg body weight such as 1 to 40 mg/kg body weight. Again, a physician will be able to determine the required route of administration and dosage for any particular patient.

The following definitions are provided to facilitate understanding of certain terms used frequently herein before.

A “transgene” comprises a polynucleotide, isolated from nature, which has been manipulated in-vitro and can then subsequently be introduced into the genome of the same or different species in either the native or modified forms, such that it is stably and heritably maintained in that genome. A “Chimaeric transgene” comprises a polynucleotide created or derived from both a human and a non-human animal polynucleotide sequence, which has been been manipulated in-vitro and can then subsequently be introduced into the genome of the host non-human animal in either the native or modified forms, such that it is stably and heritably maintained in that genome. In either case native forms include unmodified polynucleotides isolated from a source different to that into which it is subsequently introduced. Modified polynucleotides include those which have one or more nucleotide substitutions, deletions, insertions or inversions. Native or modified polynucleotides may be operably linked to a heterologous promoter, or other regulatory sequence, from a different gene within the same species or from a gene in a different species. A polynucleotide is operably linked to a regulatory sequence when, for example, it is placed under the transcriptional control of said regulatory sequence. The polynucleotide may or may not encode a polypeptide, and if a polypeptide is expressed from the polynucleotide, said polypeptide may or may not be full-length relative to that encoded by the original polynucleotide isolated. The term transgene is generally used to refer to the polynucleotide and the regulatory sequences to which it is operably linked but may refer to the polynucleotide minus any regulatory sequences.

An organism into which a transgene has been introduced is termed a “transgenic” organism.

“Regulatory sequences” refer to DNA or RNA polynucleotide sequences, which are usually non-coding, that are involved in the regulation of transcriptional activity or tissue-specific enhancement or silencing of gene transcription. Such regulatory sequences include promoters and enhancers.

“Identity” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, N.Y., 1988; Biocomputing: Informatics and Genome Projects, Smith, D. W., ed., Academic Press, N.Y., 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, N.Y., 1991; and Carillo, H., and Lipman, D., SIAM J. Applied Math., 48:1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1):387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol Biol. 215: 403-410 (1990). The well known Smith Waterman algorithm may also be used to determine identity.

Preferred parameters for polypeptide sequence comparison include the following:

1) Algorithm: Needleman and Wunsch, J. Mol Biol. 48: 443-453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Preferred parameters for polynucleotide comparison include the following:

1) Algorithm: Needleman and Wunsch, J. Mol Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

By way of example, a polynucleotide sequence of the present invention may be identical to the reference sequence of SEQ ID NO:1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:1 by the numerical percent of the respective percent identity (divided by 100) and subtracting that product from said total number of nucleotides in SEQ ID NO:1, or:
nₙ≦xₙ−(x_{n●y}),
wherein nₙ is the number of nucleotide alterations, xₙ is the total number of nucleotides in SEQ ID NO:1, and y is, for instance, 0.70 for 70%, 0.80 for 80%, 0.85 for 85%, 0.90 for 90%, 0.95 for 95%,etc., and wherein any non-integer product of xₙ and y is rounded down to the nearest integer prior to subtracting it from xₙ. Alterations of a polynucleotide sequence encoding the polypeptide of SEQ ID NO:4 may create nonsense, missense or frameshift mutations in this coding sequence and thereby alter the polypeptide encoded by the polynucleotide following such alterations.

Similarly, a polypeptide sequence referred to herein may be identical to the reference sequence of SEQ ID NO:4, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:4 by the numerical percent of the respective percent identity (divided by 100) and then subtracting that product from said total number of amino acids in SEQ ID NO:4, or:
nₐ≦xₐ−(x_{a●y})
wherein nₐ is the number of amino acid alterations, xₐ is the total number of amino acids in SEQ ID NO:4, and y is, for instance 0.70 for 70%, 0.80 for 80%, 0.85 for 85% etc., and wherein any non-integer product of xₐ and y is rounded down to the nearest integer prior to subtracting it from x_{a.}

The invention is now illustrated by reference to the following Examples.

### EXAMPLES

1. Generation of a Functional Chimaeric Human/Murine ICAM-1 cDNA.

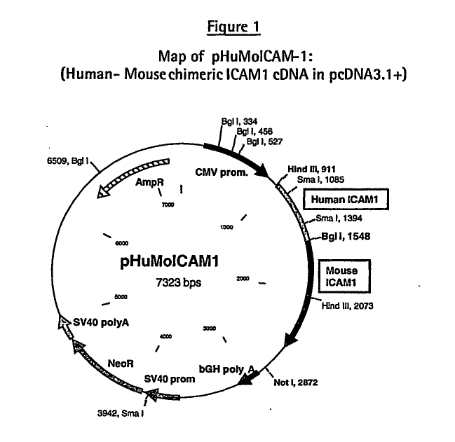

The engineering of a chimaeric ICAM-1 cDNA was facilitated by a conserved Bgl I restriction site located between domains 2 and 3 of both the human and murine ORFs (Seq ID no:14). Plasmids pCDM8-HuICAM-1 and pBS-Mo-ICAM-1 ( Siu, G., et.al, J.Immunol. 143, 3813-3820, 1989) containing the full-length cDNAs of human and murine ICAM-1, respectively, were provided by A. Craig (University of Oxford) and D. Haskard (Royal Postgraduate Medical School, London) respectively. Hind III to Not I fragments for each ORF were subcloned into pCDNA3.1 (+) (Invitrogen). The Hind III to BgI I fragment containing murine ICAM-1 domains 1 & 2 was replaced by a Hind III-Bgl I fragment containing human ICAM-1 domains 1 & 2 to create pHu/MuICAM-1 (as shown in FIG. 1). Reading frame integrity was confirmed by [³⁵S] methionine-labelled in vitro translation, using a reticulocyte lysate system (Promega) followed by SDS-PAGE and autoradiography. There was no difference in the apparent size of the recombinant protein, compared to that of human or murine ICAM-1.

To further confirm the integrity and functionality of the chimaeric ICAM-1 in pHu/MuICAM-1, it was also expressed in the monkey kidney cell line Cos7, which is otherwise non permissive for entry of major group HRV. The ability of the chimaeric molecule to support binding, cell entry and replication of major goup virus was confirmed by challenge of cells with HRV16 and immunoprecipitation of virus-specific proteins from lysates. This was followed by growth of live virus from supernatants of transfected but not mock-transfected cells.

Domain replacement had no effect on the apparent size of the protein when translated in vitro and visualized by SDS PAGE and preliminary in vitro data suggest that intracellular signalling through the chimaeric molecule is broadly equivalent to that through murine ICAM-1.

2. Generation of STable Cell Lines Expressing Chimaeric Hu/MuICAM-1

Subconfluent monolayers of murine respiratory epithelial cells (LA-4) were transfected with pHu/MuICAM-1 cDNA using Lipofectin (Life Technologies) according to the manufacturer's recommendations. Stably transfected LA-4 cells were selected in the presence of the antibiotic G418˜600,μg/ml with subsequent maintenance at 250μg/ml.

The ability of these cells to express chimaeric ICAM-1 and to support major group HRV replication was examined.

2.1 Expression of Chimaeric ICAM-1 in Mouse Respiratory Epithelial Cells

Monolayers of recombinant LA-4 cells in multi-well plates were fixed with 3% paraformaldehyde in PBS, washed with PBS and incubated with ananti-human ICAM-1 monoclonal antibody 15.2 (Serotec) at 5 μg/mL in PBS. After washing, cells were incubated with FITC-conjugated polyclonal antibodies raised against mouse immunoglobulins (Dako) at a dilution of 1:2000 in PBS. Fluorescent photomicroscopy was carried out using an Olympus UV microscope. Immunofluorescence data confirmed expression of chimaeric ICAM-1 in recombinant LA-4 cells.

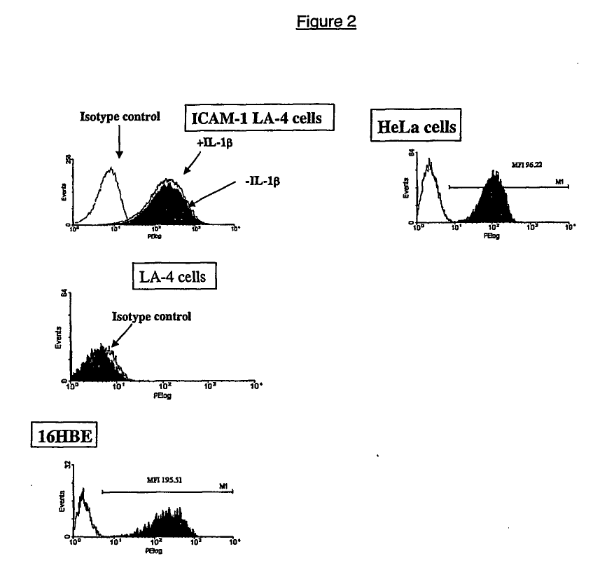

Further confirmation of the expression of chimaeric ICAM-1 in recombinant LA-4 cells was provided by flow cytometric (FACS) analysis of the transfected cell line with a species specific antibody for human ICAM-1. For the analysis, approximately 2×10⁵ cells were washed in PBS supplemented with 2% FCS and 0.01% NaN₃ and incubated with 10 μl of anti-ICAM-1 R-phycoerythrin (PE)-conjugated monoclonal antibody HA58 or its isotype control (Becton Dickinson) at 4° C. for 30 min. Cells were washed three times, resuspended in PBS and analysed for fluorescence by single color flow cytometry using a Beckman Coulter EPICS ELITE™. The flow-cytometry was used to compare the expression of ICAM-1 in the stably transfected cells (LA4-Hu/MuICAM-1), the LA4 parental cell line, a human bronchial epithelial cell line (16HBE) and the human cell line Ohio HeLa, used for routine propagation of HRV's. The analysis (FIG. 2) shows that recombinant LA-4-Hu/Mu ICAM-1 cells express chimaeric ICAM-1 at similar levels as human ICAM-1 in 16 HBE and Ohio HeLa cells

2.2 Major Group HRV Infection of Mouse Respiratory Epithelial Cells

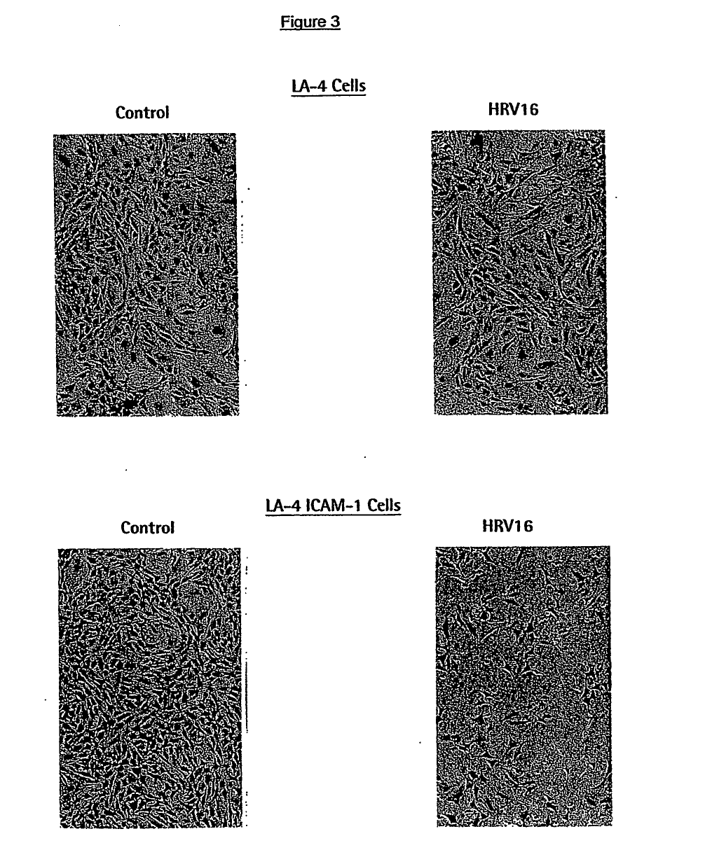

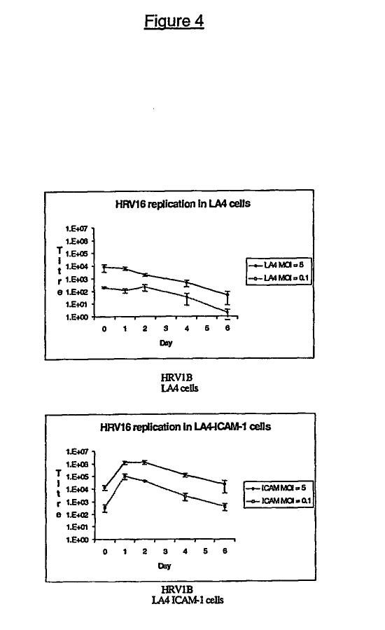

LA-4 parental (control) and stably transfected (LA4-ICAM-1) cells were challenged with major group HRV16 at high (5) and low (0.1) MOI. The appearance of the cells and virus titre of cell extracts were recorded at various time points post-infection. Challenge with HRV16 induced no cytopathic effect (CPE) in the LA-4 control cells (FIG. 3). Furthermore, after inoculation of these cells with HRV1 6 virus titres declined (FIG. 4) at the same rate as that observed for the decay of virus in medium lacking cells. This further confirmed the inability of these cells to support the replication of major group HRV.

However, after inoculation of LA-4-Hu/Mu ICAM-1 cells with HRV16, the cells developed cytopathic effects (CPE) (FIG. 3) and supported viral replication judged by rising viral titres as measured in a virus plaque assay shown in FIG. 4. Major group HRV16 replicated only in those murine respiratory epithelial cells expressing the chimaeric ICAM-1, demonstrating that expression of the correct ICAM-1 molecule capable of virus binding is both necessary and sufficient for efficient major group HRV infection of these cells.

3. Production of Transgenic Animals Expressing Chimaeric ICAM-1

Mice, transgenic for the Hu/Mu chimaeric ICAM-1 construct, were generated using the process of ES -cell mediated transgenesis, as described below:

3.1 Transgene Preparation

25 μg microgrammes of pHu/Mu ICAM-1 (see FIG. 1,) were digested with the Sal1 restriction endonuclease, which cuts at base pair (bp) positions 32 and 5133 on the plasmid, to yield 5101 bp and 2222 bp fragments respectively. Digestion was confirmed by agarose gel electrophoresis, and the 5101 bp fragment, containing the CMV promoter, chimaeric Hu/Mu ICAM, bovine growth hormone poly adenylation sequence (bGHpA) and the SV40 promoter, Neo resistance gene, SV40polyA expression cassettes, was purified using a Qiaquick™ kit (Promega). The DNA was precipitated, washed and the pellet resuspended in 25 μL phosphate-buffered saline (PBS) solution, according to standard methodolgies (Maniatis et. al., 1982, Molecular Cloning. A Laboratory Manual, Published by Cold Spring Harbor, Laboratory Press, N.Y.)

3.2 Culture and Generation of Recombinant Embryonic Stem Cell (ES) Cell Lines

HM-1 embryonic stem cells (Magin M., McWhir, J. and Melton, D., in Nucleic Acids Res. 20 (14) 3795,1992), a feeder-independent ES cell-line derived from the 129P2/Ola Hsd mouse, were used in this study. The cells were routinely cultured in “ES cell medium”; BHK21 (Glagow's medium, GIBCO) supplemented with 0.1 mM non-essential amino acids (100X stock, Gibco Cat. No320-1140AG), 10⁻⁶ mM sodium pyruvate (100X stock, Gibco Cat.320-1360AG), 10⁶ M β-Mercaptoethanol (100X stock stored at −20° C., Sigma Cat.No.600564AG), 50 μgml⁻¹ Gentamicin (50 mg ml⁻¹ stock, Gibco Cat. No. 15750-045), Lif ESGRO™ (10⁷ Units ml⁻¹ stock, Chemicon International, Cat. No. ESG1107) and 10% Foetal Calf Serum (FCS, Tissue Culture Services, Buckingham, UK). Once prepared, the medium was filtered through a 0.2μfilter (type CA, Nalgene). Culture dishes were treated for 15 minutes prior to use, with 0.1% gelatin (Sigma) in Phosphate Buffered Saline solution (PBS, NaCl 8.0 g, KCI 0.2 g, KH₂PO₄ 0.2 g and Na₂KPO₄ 1.15 g in 1 litre at pH 7.2). Cells were grown in the absence of mouse embryonic fibroblast feeder cells at 37° C. and in an atmosphere of 95% humidity, 5% CO_{2.} For routine maintenance, the cells were passaged every 3-4 days and replated on gelatinised dishes.

To create stable, recombinant ES cell lines, approximately 2×10⁷ cells in 0.8 ml PBS were mixed with 25 μg of linear pHu/Mu ICAM-1 (as described above) in a 0.4 cm electroporation cuvette (BioRad) and electroporated at 500 μF, 260 Volts at room temperature. Following electroporation cells were plated at different densities on 5×10 cm cell culture dishes (Nunc). After 24 hours the cells were washed and fed with new medium and then at 48hours, placed under positive selection (G418 antibiotic at 300 μg ml⁻¹). Cells were washed and fed every two days with fresh medium containing antibiotic. Between days 3-10, non-resistant cells died, with the subsequent appearance of colonies of G418 resistant clones from around days 8 -12.

To expand the resistant clones, individual G418-resistant colonies were “picked-off” from the 10 cm dishes using a sterile p20 pipette and tip (Gilson) set at 10 μl. The colony was picked into a 50 μl droplet of Trypsin-EDTA (Cat. No. 043-05300M), in a sterile petri dish, pipetted up and down 2-3 times and subsequently transferred to 2 ml of fresh ES cell medium plus antibiotic in a single well of a 24 well plate. In this manner, 48 G418 resistant clones were picked and expanded into individual wells of two 24-well plates. After approximately 10-14 days, when the colonies on each 24-well plate became confluent, each well was passed into two single wells on two 24 well plates, thereby creating two identical wells each on a separate plate. The cells were again allowed to grow to near confluency and one duplicate plate (the “Master”), was frozen down, whilst the other (the “Analysis” plate) was used to provide cells for DNA preparation and analysis (See below). For freezing, the media in each well of the Master plate was aspirated and replaced with 300 μl of freezing mix (70% ES cell media, 20% Foetal Calf Serum, 10% cell culture grade DMSO), the plate placed in an insulated polystyrene box and allowed to cool gradually to −70° C. Subsequently, the plate was transferred to a −135° C. freezer for storage whilst DNA from the Analysis plate was being prepared and tested for the presence of the integrated transgene.

3.3 DNA Preparation and Screening of Recombinant Embryonic Stem (ES) Cell Lines

Genomic DNA was prepared from the ES cells by aspirating the medium from each well of the 24 well “Analysis” plate, washing once with 1 ml PBS and subsequent addition of 0.5 ml lysis buffer (100 mM Tris.HCI pH8.5, 5 mMEDTA, 0.2% SDS, 200 mMNaCl and 100 μg ml⁻¹ Proteinase K). The plate was left to shake slowly on a rotary shaker at room temperature overnight to facilitate lysis and DNA release from the cells. The DNA was precipitated by adding an equal volume of isopropanol, spun, washed in 70% ethanol solution and the precipitate resuspended in approximately 150 ml of TE buffer (10 mM Tris. HCl, 1 mM EDTA , pH 8.0). One microlitre of this DNA was used for subsequent analysis by Polymerase Chain Reaction (PCR) for the presence of the transgene in the ES cell clone. PCR was performed with the primers NS 25 (GGGCAGTCAACAGCTAAAACCT) and NS 26 (TCCAGGGAGCAAAACAACTTCT) using AmplitTaq DNA polymerase (Perkin Elmer Technologies). The PCR reactions were carried out on a Robocycler™ (Stratagene) and 30 cycles of DNA amplification at 94° C. for 40 s, 54° C. for 40 s and 72° C. for 90 s to generate a 425 bp amplicon. Greater than 50% of the ES cell clones analysed were positive for the presence of the transgene and were used to establish recombinant ES cell lines from which founder mice could be generated.

3.4 Generation of Founders and Screening of Transgenic Mice

Chimaeric (“founder”, F₀) mice were generated by blastocyst microinjection of ES cells described above, essentially as described, (Hogan, B., Costatini, F. and Lacy, E., 1986, Manipulating the mouse embryo. Cold Spring Harbor Laboratory and, Gene Targeting-A Practical Approach, edited by A. L. Joyner, 1993, 2002, Oxford University Press. Recipient blastocysts were obtained from C57 BL/6 J females, which had previously been mated with stud C57bl/6 males. Chimaeric mice were identified by the presence of the coat colour marker from the donor ES cell line (chinchilla) against the background of the recipient strain (Black). Chimaeric males were test bred by backcrossing to C57 BL/6 J females to ascertain the contribution of the ES cell line to the germline and to establish transgenic lines. In general, the degree of coat colour chimaerism of a particular chimaera correlated with the degree of germline contribution Transmission of the Hu/MuICAM-1 transgene was confirmed by PCR analysis, according to the specific assay described above. Subsequently, offspring were bred onto the Balb/c genetic background by successive backross matings (to N=10) of male transgenic mice to wild type Balb/c females.

3.5 Analysis of Chimaeric ICAM-1 Expression

Expression of chimaeric ICAM-1 in transgenic mice was analysed by Western Immunoblotting and immunohistochemistry using human ICAM-1 specific antibodies.

3.5.1 Generation of Purified Human ICAM-1 Specific Antibodies

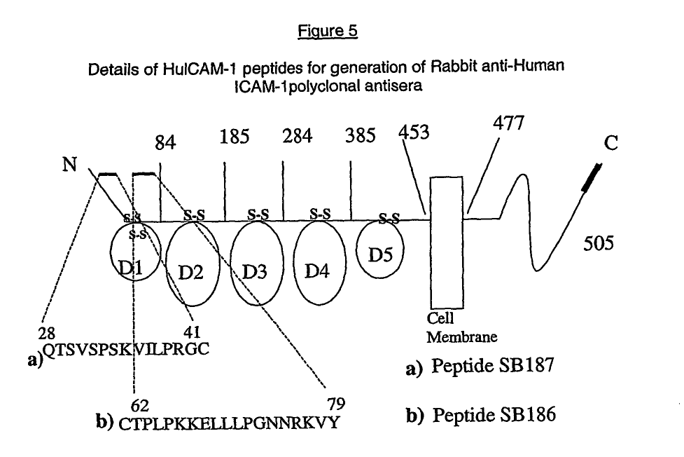

Antigenic regions of the human ICAM domain were predicted using algorithms within the Lasergene suite of programs. Two regions which are unique to human ICAM (Genpept P05362), and not present in the murine orthologue (Genpept A45815) by extensive BLAST analysis, were chosen as immunogens (see FIG. 5). The synthetic peptides SB186 CTPLPKKELLLPGNNRKVY and SB187 QTSVSPSKVILPRGC were produced using standard solid phase techniques and analysed using HPLC and MS. The peptides were conjugated to the carrier protein PPD (purified protein derivative of Tuberculin), via the Cys residues, using the cross-linker sulpho-SMCC (Perbio). The resulting conjugates were dissolved in PBS and stored at −20° C, until used.

Each peptide-carrier conjugate was used to immunise pairs of female New Zealand White rabbits. Animals were primed by innoculation of ca. 100ug conjugate emulsified in Complete Freund's adjuvant at 3 subcutaneous sites. Boosters were given at 3-4 week intervals using incomplete adjuvant. The immune responses of the animals were followed by analysis of test bleeds taken 7 days after each immunisation. The titres of each antisera was determined using indirect ELISA with microtitre plates coated with free peptide as antigen. After 3 immunisations, all rabbits had achieved a titre of >450,000 by ELISA and samples of the serum were then affinity isolated and tested for cross-reactivity with the holoprotein by immunocytochemistry, immunohistochemistry and immunoblotting. Once satisfactory results were obtained in these assays (after 3-4 immunisations) terminal bleeds were performed.

Serum samples of up to 2ml were diluted 5 fold with PBS and applied to precycled 2 ml columns (Sulpholink, Perbio) of appropriate immobilised peptide (2 mg). Unbound material was removed by washing with 10-20 volumes of PBS. Specific anti-peptide antibodies were eluted from the columns using 0.1 M glycine-HCL pH 2.5 that was neutralised by collecting into 1 M Tris pH 8.0. Purified antibodies were dialysed exhaustively into PBS and quantified by measuring absorbance at A280 nm. Samples of each antibody were stored at −80 C. until used, after adding sodium azide as preservative and crystalline BSA as stabiliser. Effective absortion and elution was confirmed in each case by analysis of flow-through and eluted materials using the indirect ELISA.

3.5.2 Analysis of Chimaeric ICAM-1 Transgene Expression by Western Immoblotting

The lung tissues were extracted from mice of approximately 3-6 months of age (N=3 backcross to Balb/c) and a portion of the right lobe taken from male and female transgenic and non-transgenic mice, snap-frozen in liquid nitrogen and stored at −80° C. until required. Tissue from each was prepared for, and analysed by Western Immunoblotting. Cell (10 μl) or tissue extracts (50 μg) were pre-treated at 100° C. in 2×DTT sample buffer without loading dye. Extracts were clarified by brief centrifugation and protein concentrations estimated using the Bradford method by reference to a BSA standard curve (Perbio). The samples were electrophoresed using BioRad Criterion 4-20% gradient gels and resolved proteins transferred to nitrocellulose filters by semi-dry blotting. The efficiency of transfer was judged using Ponceau S stain and then remaining protein binding sites on the blots saturated with 5% dried milk powder (Marvel) dissolved in PBS. Duplicate blots were made and probed with antisera raised to either, HuICAM-1 peptide SB186 (A258) or HuICAM-1 peptide SB187 (A270) at 5 μg/μl in blocking solution containing 0.1% (v.v.) tween 20 overnight at 4° C. and bound antibodies revealed using anti-rabbit immunoglobulin HRP conjugate followed by ECL detection.

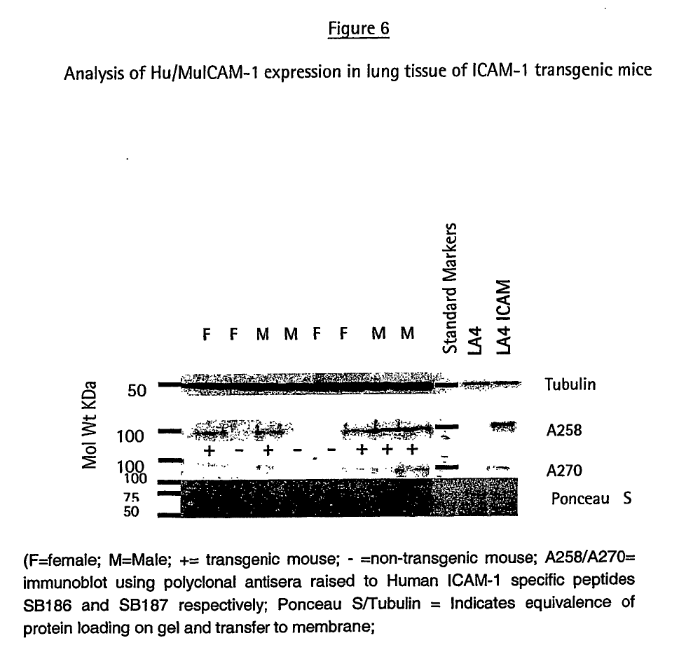

This experimental approach demonstrated that chimaeric ICAM-1 expression was observed in the lung tissue of both male and female transgenic (but not non-transgenic) mice in 50% of the transgenic lines generated. Identical results were obtained on probing with antisera raised against either peptide, thereby providing further validation and confirmation of the data. After probing for chimaeric ICAM-1 expression, the blots were stripped and reprobed using a monclonal antibody against alpha tubulin. These results confirmed approximately equivalent loadings between the different samples tested. Representative immunoblotting data is shown in FIG. 6.

3.5.3 Analysis of Chimaeric ICAM-1 Expression by Immunohistochemistry

a) Preparation of Histological Sections

Samples of nasal tissue (anterior portion of the nose) and individual lung lobes were removed, embedded in OCT compound and snap frozen using isopentane/dry ice. Frozen sections (6 μm thick) of nasal airway and lung were prepared using a cryostat from a total of eight mice, two transgenic i.e, 1 male and 1 female positive (as determined by PCR analysis of DNA obtained from tail-biopsy samples) and 1 male and 1 female negative transgenic littermate control, from each of two transgenic lines. In each case, the nasal airways and lung tissue were sectioned. Each section was post fixed in ice-cold 4% paraformaldehyde for 5 minutes prior to immunohistochemistry.

b) Immunocytochemical Staining

Immunohistochemistry was performed on an ‘Optimax’ automatic immunostaining machine (BioGenex) using routine methods. The sections were incubated with the primary antibody or the corresponding pre-immune serum, for 1 hour at room temperature after which they were then incubated with goat ant-rabbit secondary antiserum labelled with HRP strepavidin. Finally, positive staining was visualised using standard DAB detection. Initial experiments using a fresh ampoule of the purified ICAM-1 specific antibody (prepared as hereinbefore described) at a dilution of 1:100 demonstrated background staining with the pre-immune serum. This was overcome by re-optimising the new ampoule of antibody at a dilution of 1:400. The protein concentrations of the neat antisera were 114 μg/ml; pre-immune, 134μg/ml, respectively.

The antibody (at a dilution of 1:400) was applied to a frozen section of human asthmatic lung, in order to demonstrate hICAM-1 expression in human lung with this antibody for comparison with the murine lung results. All the slides were assessed qualitatively using a light microscope for evidence of positive staining for hICAM-1.

Immunohistochemistry with the polyclonal anti-hICAM-1 antibody demonstrated moderate to strong expression of hICAM-1 in the epithelium lining the nasal airways of positive male and female hICAM-1 transgenic mice. In contrast, using the same dilution (1:400) of the antibody, no evidence of staining was detected in the nasal tissues of either male or female negative control (i.e, non transgenic) littermates. Similar results were found in the lung tissues of positive and negative mice from each of two transgenic mouse lines examined.

In the nasal airway sections of the positive transgenic animals, cell types that stained positive with the antibody included epithelium, smooth muscle and various unidentified cells of the submucosa. Similarly in the lungs of the positive transgenics, strong hICAM-1 staining was detected in alveolar macrophages as well as the airway epithelium, with weaker staining present in airway and vascular smooth muscle. Moreover, the staining pattern observed in the lungs of the positive transgenic mice was identical to that seen in human asthmatic lung.

In conclusion the immunohistochemistry using the polyclonal antibody to domains 1 and 2 of hICAM-1 demonstrated positive expression of hICAM-1 in the epithelium lining the nasal passages and the pulmonary airways of two lines of hICAM-1 transgenic mice tested, thereby further validating these animals as suitable models for HRV infection studies.

3.6 HRV-induced Inflammatory Cytokine Production and Cell Necrosis in Primary Tracheal Cells Isolated from Transgenic Mice

Trachea were harvested from transgenic and non-transgenic littermate control mice and digested at 37° C. with pronase ( 0.25 mg/ml) and Dnase1 (0.2 mg/ml) in 10 ml PBS (without Ca²⁺ or Mg²⁺) for 30-45 minutes. The suspension was stirred continuously using a sterile magnetic stirrer bar and magnetic stirrer until clumps of cells sloughing off the tissue were visible. The protease was neutralised by adding 10% Fetal calf serum (FCS) and the tissue remains were removed. The remaining suspension was spun at 1100 rpm for 5 minutes and the cell pellet resuspended as required. After preplating the digested mixture on tissue culture dishes, to selectively remove monocytic cells by adherence, the supernate was transferred to dishes coated in collagen. Collagen coated dishes were prepared as follows; collagen type 1 from rat tail (Upstate Biotechnology, cat. No. #08-115) was diluted with PBS to 10 ug/ml and added to cover the surface of the cell culture dish. After incubation at room temperature for 10-30 minutes followed by aspiration, the collagen coated dishes were washed with PBS or medium. When confluent, the primary epithelial cell cultures were infected with HRV 16 at 1 m.o.i for 48 hours before measurements of inflammatory cytokines (IL6, RANTES) and cell necrosis (LDH release) were taken. For IL6 and RANTES, levels were measured on ELISA using commercially available kits (R & D Systems) and for LDH release a Cytotox96™ non radioactive cytotoxicity assay (Promega, cat. No. G1781) was used.

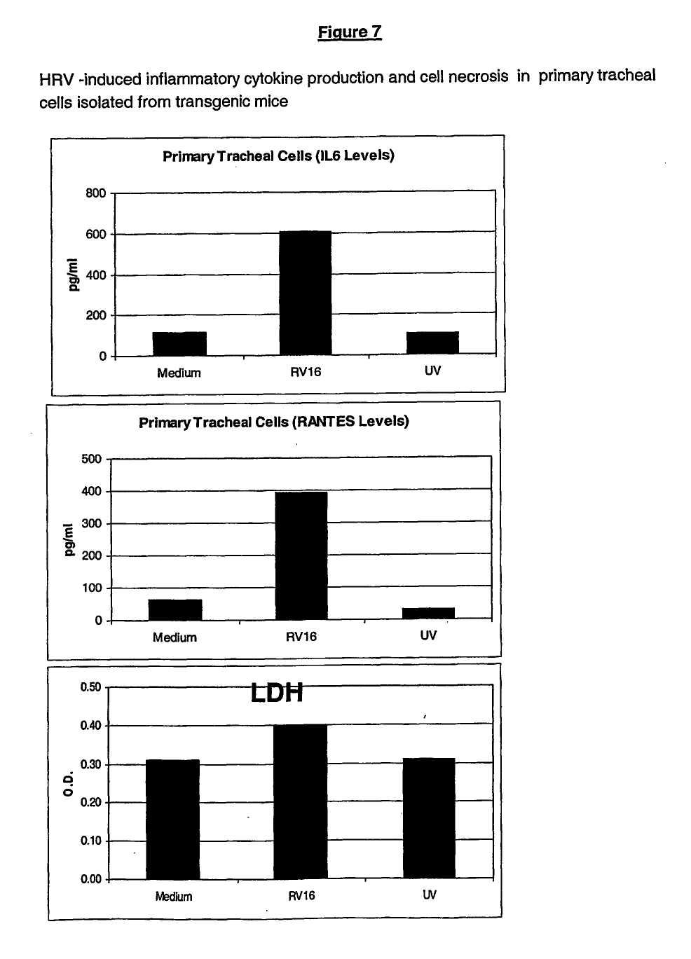

Representative data can be seen in FIG. 7. The levels of IL6 and RANTES were significantly raised in primary tracheal epithelial cells, infected with HRV, from transgenic mice compared to non transgenic mice or mice treated with medium alone. Also, primary tracheal cells from transgenic mice, infected with UV inactivated virus showed no increase in IL6 or RANTES production. Finally, cell death due to cytopathic effects (CPE), as measured by LDH release, was elevated significantly in HRV treated transgenic primary cells, but not in cells treated with medium or inactivated virus. These results are strongly indicative of HRV infection in primary tracheal cells isolated from the transgenic, but not non-transgenic mice, and further demonstrates of the ability of the transgenic mice of the invention to support HRV infection in cells and tissues of the respiratory tract.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

What is claimed is:

1. A transgenic mouse whose genome comprises a polynucleotide encoding human ICAM-1 domains D1 and D2.

2. A transgenic mouse according to claim 1, wherein said polynucleotide encodes human ICAM-1 domains D1 and D2 and one or more mouse ICAM-1 domains D3, D4 or D5.

3. A transgenic mouse according to claim 2, wherein said polynucleotide encodes human ICAM-1 domains D1 and D2 and mouse ICAM-1 domains D3, D4 and D5.

4. A transgenic mouse according to claim 1, wherein said polynucleotide comprises one or more of the following polynucleotide sequences:
(a) a polynucleotide sequence having at least 90% sequence identity to the polynucleotide sequence of SEQ ID No:2;
(b) a polynucleotide sequence having at least 90% sequence identity to the polynucleotide sequence of SEQ ID No:3;
(c) a polynucleotide sequence encoding a polypeptide sequence having at least 90% sequence identity to the polypeptide sequence of SEQ ID No:5;
(d) a polynucleotide sequence encoding a polypeptide sequence having at least 90% sequence identity to the polypeptide sequence of SEQ ID No:6;
(e) a polynucleotide fragment of SEQ ID No:1 encoding human ICAM-1 domains D1 and D2; and
(f) a polynucleotide sequence encoding a polypeptide fragment of SEQ ID No:4 comprising human ICAM-1 domains D1 and D2.

5. A transgenic mouse according to claim 1, whose genome further comprises a regulatory sequence capable of directing expression of said polynucleotide in cells of the respiratory tract.

6. A transgenic mouse according to claim 5, wherein said regulatory sequence is a promoter selected from the group consisting of CMV, SV40, human surfactant protein C (SPC) or Clara cell 10 KDa secretory (CC10).

7. A transgenic mouse according to claim 1, which expresses an ICAM-1 polypeptide comprising human ICAM-1 domains D1 and D2.

8. A transgenic mouse according to claim 7, which expresses a chimaeric ICAM-1 polypeptide comprising human ICAM-1 domains D1 and D2.

9. A transgenic mouse according to claim 8, which expresses a chimaeric ICAM-1 polypeptide comprising one or more of the following polypeptide sequences:
(a) a polypeptide fragment of SEQ ID No:4 comprising human ICAM-1 domains D1 and D2;
(b) a polypeptide sequence having at least 90%, sequence identity with the polypeptide sequence of SEQ ID No:5; and
(c) a polypeptide sequence having at least 90%, sequence identity with the polypeptide sequence of SEQ ID No:6.

10. A transgenic mouse according to claim 9, wherein said chimaeric ICAM-1 polypeptide comprises one or more of mouse ICAM-1 domains D3, D4 and D5.

11. A transgenic mouse according to claim 10, wherein said chimaeric ICAM-1 polypeptide comprises mouse ICAM-1 domains D3, D4, and D5.

12. A transgenic mouse according to claim 8, wherein said chimaeric ICAM-1 polypeptide is expressed in cells of the respiratory tract.

13. A transgenic mouse according to claim 8, wherein said chimaeric ICAM-1 polypeptide is capable of binding and supporting major group human rhinovirus (HRV) infection.

14-16. (canceled)

17. A transgenic mouse according to claim 1 wherein said polynucleotide comprises one or more of the following polynucleotide sequences:
(a) a polynucleotide sequence having at least 90%, sequence identity to the polynucleotide sequence of SEQ ID No:8;
(b) a polynucleotide sequence having at least 90%, sequence identity to the polynucleotide sequence of SEQ ID No:9;
(c) a polynucleotide sequence having at least 90%, sequence identity to the polynucleotide sequence of SEQ ID No:10;
(d) a polynucleotide sequence encoding a polypeptide sequence having at least 90% sequence identity to the polypeptide sequence of SEQ ID No:11;
(e) a polynucleotide sequence encoding a polypeptide sequence having at least 90% sequence identity to the polypeptide sequence of SEQ ID No:12;
(f) a polypeptide sequence encoding a polypeptide sequence having at least 90% sequence identity to the polypeptide sequence of SEQ ID No:13;
(g) a polynucleotide fragment of SEQ ID No:7 encoding one or more of murine ICAM-1 domains D3, D4 and D5;
(h) a polynucleotide sequence having at least 90% sequence identity to the polynucleotide sequence of SEQ ID No:14; and
(i) a polynucleotide encoding a polypeptide having at least 90% sequence identity to the polypeptide sequence of SEQ ID No:15.

18. A transgenic animal mouse according to claim 7, wherein said chimaeric ICAM-1 polypeptide comprises one or more of the following polypeptide sequences:
(a) a polypeptide fragment of SEQ ID No:7 comprising one or more of murine ICAM-1 domains D3, D4 and D5;
(b) a polypeptide sequence having at least 90% sequence identity with the polypeptide sequence of SEQ ID No:11;
(c) a polypeptide sequence having at least 90% sequence identity with the polypeptide sequence of SEQ ID No:12;
(d) a polypeptide sequence having at least 90% sequence identity with the polypeptide sequence of SEQ ID No:13; and
(e) a polypeptide sequence having at least 90% sequence identity to the polypeptide sequence of SEQ ID No:15.

19-22. (canceled)

23. An isolated polynucleotide comprising a polynucleotide sequence encoding human ICAM-1 domains D1 and D2 and comprising a regulatory sequence capable of directing expression of said polynucleotide in cells of the murine respiratory tract.

24. A vector comprising a polynucleotide according to claim 23.

25. A cell stably transfected with a polynucleotide according to claim 23.

26. (canceled)

27. A method of producing a transgenic mouse comprising introducing a polynucleotide according to claim 23 into murine ES cells using electroporation, retroviral vectors or lipofection for gene transfer.

28. (canceled)

29. A method of screening test agents for use in the treatment of a condition associated with major group HRV infection, the method comprising administering a test agent to a transgenic mouse according to claim 1 and determining whether the test substance (i) prevents or delays the onset of the condition or (ii) alleviates the condition.


SEQUENCE LISTING (20 sequences)

SEQ ID NO: 1 - DNA, 2984 bp, Homo sapiens
gcgccccagtcgacgctgagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc

SEQ ID NO: 2 - DNA, 189 bp, Homo sapiens
ggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagct

SEQ ID NO: 3 - DNA, 198 bp, Homo sapiens
ggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcgg

SEQ ID NO: 4 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 5 - protein, 63 aa, Homo sapiens
GGSVLVTCSTSCDQPKLLGIETPLPKKELLLPGNNRKVYELSNVQEDSQPMCYSNCPDGQSTA

SEQ ID NO: 6 - protein, 66 aa, Homo sapiens
GKNLTLRCQVEGGAPRANLTVVLLRGEKELKREPAVGEPAEVTTTVLVRRDHHGANFSCRTELDLR

SEQ ID NO: 7 - DNA, 1614 bp, Mus sp.
atggcttcaacccgtgccaagcccacgctacctctgctcctggccctggtcaccgttgtgatccctgggcctggtgatgctcaggtatccatccatcccagagaagccttcctgccccagggtgggtccgtgcaggtgaactgttcttcctcatgcaaggaggacctcagcctgggcttggagactcagtggctgaaagatgagctcgagagtggacccaactggaagctgtttgagctgagcgagatcggggaggacagcagtccgctgtgctttgagaactgtggcaccgtgcagtcgtccgcttccgctaccatcaccgtgtattcgtttccggagagtgtggagctgagacctctgccagcctggcagcaagtaggcaaggacctcaccctgcgctgccacgtggatggtggagcaccgcggacccagctctcagcagtgctgctccgtggggaggagatactgagccgccagccagtgggtgggcaccccaaggaccccaaggagatcacattcacggtgctggctagcagaggggaccacggagccaatttctcatgccgcacagaactggatctcaggccgcaagggctggcattgttctctaatgtctccgaggccaggagcctccggactttcgatcttccagctaccatcccaaagctcgacacccctgacctcctggaggtgggcacccagcagaagttgttttgctccctggaaggcctgtttcctgcctctgaagctcggatatacctggagctgggaggccagatgccgacccaggagagcacaaacagcagtgactctgtgtcagccactgccttggtagaggtgactgaggagttcgacagaaccctgccgctgcgctgcgttttggagctagcggaccagatcctggagacgcagaggaccttaacagtctacaacttttcagctccggtcctgaccctgagccagctggaggtctcggaagggagccaagtaactgtgaagtgtgaagcccacagtgggtcgaaggtggttcttctgagcggcgtcgagcctaggccacccaccccgcaggtccaattcacactgaatgccagctcggaggatcacaaacgaagcttcttttgctctgccgctctggaggtggcgggaaagttcctgtttaaaaaccagaccctggaactgcacgtgctgtatggtcctcggctggacgagacggactgcttggggaactggacctggcaagaggggtctcagcagactctgaaatgccaggcctgggggaacccatctcctaagatgacctgcagacggaaggcagatggtgccctgctgcccatcggggtggtgaagtctgtcaaacaggagatgaatggtacatacgtgtgccatgcctttagctcccatgggaatgtcaccaggaatgtgtacctgacagtactgtaccactctcaaaataactggactataatcattctggtgccagtactgctggtcattgtgggcctcgtgatggcagcctcttatgtttataaccgccagagaaagatcaggatatacaagttacagaaggctcaggaggaggccataaaactcaagggacaagccccacctccctga

SEQ ID NO: 8 - DNA, 204 bp, Mus sp.
ggcacccagcagaagttgttttgctccctggaaggcctgtttcctgcctctgaagctcggatatacctggagctgggaggccagatgccgacccaggagagcacaaacagcagtgactctgtgtcagccactgccttggtagaggtgactgaggagttcgacagaaccctgccgctgcgctgcgttttggagctagcggaccag

SEQ ID NO: 9 - DNA, 162 bp, Mus sp.
gggagccaagtaactgtgaagtgtgaagcccacagtgggtcgaaggtggttcttctgagcggcgtcgagcctaggccacccaccccgcaagtccaattcacactgaatgccagctcggaggatcacaaacgaagcttcttttgctctgccgctctggaggtg

SEQ ID NO: 10 - DNA, 159 bp, Mus sp.
gaggggtctcagcagactctgaaatgccaggcctgggggaacccatctcctaagatgacctgcagacggaaggcagatggtgccctgctgcccatcggggtggtgaagtctgtcaaacaggagatgaatggtacatacgtgtgccatgcctttagctcc

SEQ ID NO: 11 - protein, 68 aa, Mus sp.
GTQQKLFCSLEGLFPASEARIYLELGGQMPTQESTNSSDSVSATALVEVTEEFDRTLPLRCVLELADQ

SEQ ID NO: 12 - protein, 54 aa, Mus sp.
GSQVTVKCEAHSGSKVVLLSGVEPRPPTPQVQFTLNASSEDHKRSFFCSAALEV

SEQ ID NO: 13 - protein, 53 aa, Mus sp.
EGSQQTLKCQAWGNPSPKMTCRRKADGALLPIGVVKSVKQEMNGTYVCHAFSS

SEQ ID NO: 14 - DNA, 1608 bp, Artificial sequence
  DNA encoding a human/mouse chimaeric ICAM-1 polypeptide
atggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggcattgttctctaatgtctccgaggccaggagcctccggactttcgatcttccagctaccatcccaaagctcgacacccctgacctcctggaggtgggcacccagcagaagttgttttgctccctggaaggcctgtttcctgcctctgaagctcggatatacctggagctgggaggccagatgccgacccaggagagcacaaacagcagtgactctgtgtcagccactgccttggtagaggtgactgaggagttcgacagaaccctgccgctgcgctgcgttttggagctagcggaccagatcctggagacgcagaggaccttaacagtctacaacttttcagctcctgtcctgaccctgagccagctggaggtctcggaagggagccaagtaactgtgaagtgtgaagcccacagtgggtcgaaggtggttcttctgagcggcgtcgagcctaggccacccaccccgcaagtccaattcacactgaatgccagctcggaggatcacaaacgaagcttcttttgctctgccgctctggaggtggcgggaaagttcctgtttaaaaaccagaccctggaactgcacgtgctgtatggtcctcggctggacgagacggactgcttggggaactggacctggcaagaggggtctcagcagactctgaaatgccaggcctgggggaacccatctcctaagatgacctgcagacggaaggcagatggtgccctgctgcccatcggggtggtgaagtctgtcaaacaggagatgaatggtacatacgtgtgccatgcctttagctcccatgggaatgtcaccaggaatgtgtacctgacagtactgtaccactctcaaaataactggactataatcattctggtgccagtactgctggtcattgtgggcctcgtgatggcagcctcttatgtttataaccgccagagaaagatcaggatatacaagttacagaaggctcaggaggaggccataaaactcaagggacaagccccacctccctga

SEQ ID NO: 15 - protein, 535 aa, Artificial sequence
  Amino acid sequence of a human/mouse chimaeric ICAM-1 polypeptide
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 16 - DNA, 2602 bp, Rattus sp.
ctgctgcctgcactttgccctggtcctccaatggcttcaacccgtgccaggcccatgctgcctctgctcctggtcctggtcgccgttgtgatccccgggcctgtcggtgctcaggtatccatccatcccacagaagccttcctgcctcggggtggatccgtgcaggtgaactgctcttcctcttgcgaagacgagaacctcggcctggggttggagactaactggatgaaagacgaactatcgagtggacacaactggaagctcttcaagctgagcgacattggggaagacagcagaccactgtgctttgagaactgtggcaccacgcagtcctcggcttctgccaccatcactgtgtattcgttcccagagcgagtggagctggatcctctgcccgcctggcagcaggtgggcaagaacctcatcctgcgctgcctggtggaaggcggagcaccgcggacacagctctcagtagtgctgctccgtgggaatgagacactgagccgccaggcagtggatggggaccccaaggagatcacattcacggtgctggccagcagaggcgaccacggagccaatttctcatgcttcacagaactggacctcaggccacaagggctgtcactgttcaagaatgtctccgaggtcaggcagctccggactttcgatcttccgactagggtcctgaagctcgacacccctgacctcctggaggtgggcacccagcagaagttcttgtgttccctggaaggcctgtttcctgcctctgaagctcagatatacctggagatgggaggccagatgctgaccctggagagcacaaacagcagagattttgtgtcagccactgcctcagtggaggtgactgagaagttggacagaaccctgcagctgcgctgtgttttggagctggcggaccagaccctggagatggagaagaccttgagaatctacaacttttcagctcccatcctgaccctgagccagccggaggtctcagaaggggaccaagtaactgtgaagtgtgaagcccacggtggggcacaggtggtgcttctgaacagtacttcccccaggccacccacctcacagggtacttcccccaggccacccacctcacagatccaattcacactgaatgccagcccggaggatcacaaacgacgcttcttttgctctgcggccttggaggtggatgggaagtccctgtttaaaaaccagaccttggaactccatgtgctatatggtcctcacctggacaagaaggactgcttggggaactggacctggcaagaggggtctcagcagactcttacatgccagccccaggggaatccagcccctaatctgacctgcagccggaaagcagatggtgtcccgctgcctatcgggatggtgaagtctgtcaaacgggagatgaatggtacctacaagtgccgtgcctttagctcccgtgggagtatcaccagggacgtgcacctgacagtgctgtaccatgatcagaatacctgggtcataattgttggtgtgttggtactgatcattgcgggcttcgtgatcgtggcgtccatttacacctattaccgccagaggaagatcaggatatacaagttacagaaggctcaggaggaggccctaaaactcaaggtacaagccccgcctccctgagcccactggacaggacacctgcctgggccccgctgctcttgaacagatcaatggacagcatttacccctcacccacctcctctggctgtcacaggacaggacagtggcctggggatgcatacttgtagcctcaggcctaagaggactcggaggggcaagactgtgaactcgtgacctggacacacctacagcctggtgggcctgcagccaagaaaggctgacttccttctctattacccctgctgaggggccccctaccttaggaaggtgtgatatccggtagacacaagcaagagaagaaaaggaacaccatgcttcctctgacatgggaaagctgggacactgtccccaactcttgttgatgtatttattaattcagagttctgacagttatttattgagtaccctgtacagacactagaggagtgagcaggttaacatgtaagttattgcctagaccctggtgaaggggcacaacagagtctggggaaagatcatacgggtttgggcttctccacaggtcagggtgctttcctcaaaagagctgatttctttcacgagtcatataaatactatgtggacgagcagtggccctctgctcgtagacctctctgggacccctgcctcctcccacagcctggagtctcccagcaccagcatgggtgaccacctccccacctacatacattcctacctttgttcccaatgtcaaccaccatgcctaaatatggacgctcacctttagcagctcaacaatggagtctcatgcccgtgaaattatggtcaatccctgcatgcctccacccggctccacctcaaagagaatgcctgggagaaaatgttccaaccacttagaagggtcctgcaagctgttgtgggagggtaggcacccctcccagcgcagaagcctttcctttgaatcaataaagtttta

SEQ ID NO: 17 - protein, 19 aa, Artificial sequence [sequence omitted]

SEQ ID NO: 18 - protein, 15 aa, Artificial sequence
  Synthetic peptide SB187
QTSVSPSKVILPRGC

SEQ ID NO: 19 - DNA, 22 bp, Artificial sequence
  Primer NS 25
gggcagtcaacagctaaaacct

SEQ ID NO: 20 - DNA, 22 bp, Artificial sequence
  Primer NS 26
tccagggagcaaaacaacttct